(19) United States
(12) Patent Application Publication
(10) Pub. No.: US 2011/0135693 A1
(43) Pub. Date: Jun. 9, 2011

(54) PATCH

(76) Inventor: Steffen Barth, Obermaiselstein (DE)

(21) Appl. No.: 12/920,006

(22) PCT Filed: Feb. 20, 2009
(86) PCT No.: PCT/EP2009/001365

§ 371 Date: Dec. 15, 2010

(30) Foreign Application Priority Data
Feb. 28, 2008 (DE) 10 2008 011 566.5
Aug. 1, 2008 (DE) 10 2008 035 848.7

(51) Int. Cl. A61K 9/00; B05D 1/36

(52) U.S. Cl. 424/400; 427/2.14

(57) ABSTRACT

An adhesive patch for attaching to the skin for medical, cosmetic, and/or orthopedic purposes. The patch is formed with a film that includes a carrier material, which is provided with a therapeutically effective coating on both sides.

The invention relates to a patch to be applied onto the skin for medical, cosmetic, and/or orthopedic purposes, which comprises a carrier material that is provided with a therapeutically effective coating on both sides.

Patches are known in various versions in medicine and cosmetics. The present invention relates to patches for the transdermal administration of controlled amounts of active ingredients. In such patches, for example, the active agent may be dissolved or suspended in a polymer matrix film through which the active agent diffuses into the skin (cf. U.S. Pat. No. 4,839,174).

The known patches are suitable to only a limited extent for the treatment of regions with inflammation, for example on joints with capsule damage or so-called tennis elbow, which is generally treated by immobilization using a bandage optionally assisted by medication. Such treatments are laborious and, particularly in the event of treatment by medication, can lead to considerable problems for the body.

In order to improve the treatment of internal inflammations, DE 103 06 187 A1 discloses a patch which consists of an adhesive strip, on which an electrically conductive layer is adhesively bonded. The layer is arranged on the adhesive strip so that the circumferential outer edge of the adhesive strip is uncovered. The uncovered adhesive surface is used to fix the patch on the skin.

The known patch fulfills the demands required of it. Depending on the choice of the electrically conductive layer, however, it is limited to a relatively narrow range of action, for example inflammatory processes in a joint. Furthermore, the use of an adhesive strip onto which the coated film is then applied leads to a thickness of the patch which has negative effects on the wearing comfort for various applications. This applies for example to the treatment of capsule inflammations in the region of the foot, particularly in the region of the toes, since footwear is normally worn on the feet.

For sporting activities, this usually involves relatively tightly fitting shoes which are intended to give the wearer a good feel for contact with the ground. Furthermore, for example, high-heeled shoes are likewise designed to be very tight. For these and other applications, the known patches cannot be applied without loss of comfort on the toes since the thickness of the known patches creates a pressure point which lies precisely on the site to be treated. The treatment results are negatively affected by the pressure points; in the most extreme case reddening or even chafing can occur at the pressure points. Said problems are also encountered when wearing compression stockings.

Added to this, the known patches make it difficult to put on very tight compression stockings, or the patches are detached when putting them on.

The invention provides assistance in this regard. It is an object of the invention to provide a patch which improves the good treatment results of the patches known from the prior art and extends the range of application. In particular, the patch should be able to control the treatment process itself as a function of the biochemical situation on the patient's skin.

According to a first aspect of the invention, the patch has a film consisting of a carrier material, which is provided with a therapeutically effective coating on both sides. For use, one of the coatings is arranged closer to the patient's skin than the other.

The carrier material is for example a natural or synthetic polymer, preferably a polyester. In particular, a polyurethane matrix is also suitable as a carrier material. A polymer matrix may in particular have pores, i.e. through-openings, which allow penetration by skin perspiration from one side of the carrier material to the other. Experiments have shown that particularly in the case of a thin carrier layer with a thickness of less than 100 μm and in particular less than 20 μm, the coating arranged further away from the skin also contributes to the therapy (i.e. medical, cosmetic and/or orthopedic effect). The contribution may be imparted in one or more of the following ways:

In the case of electrically conductive coatings (at least when the layer arranged further away from the skin is electrically conductive) these influence the biochemical processes in or under the skin by electrically conductive contact and/or by a capacitive effect. For example, in the case of an electrically insulating carrier material and two electrically conductive coatings, the film forms an electrical capacitor. The effect of an electrochemical battery is achieved when different metal coatings are arranged on the opposite sides and skin perspiration can enter the carrier material as an electrolyte, as is the case when using a carrier material which has through-openings.

Thus, in particular, the healing effect of a noble metal can be exerted on the side placed further away from the skin (for example gold), even though the noble metal is separated from the skin by the carrier material. At the same time, for example, the coating consisting of silver which is arranged closer to the skin (and preferably is in contact directly with the skin or secretions of the skin, such as perspiration) can likewise exert the known healing effects of silver. Optionally, in any desired combination of coatings, a therapeutically effective substance may also be contained in the carrier material or form the carrier material.

For any type of coatings, the coating placed closer and the carrier material may dissolve at least locally under the effect of the biological substances on and in the skin, so that the coating on the far side of the film comes closer to the skin or even in contact with the skin or the substances. “Dissolving” is also intended to mean that the coating and the carrier material form tears or holes. Dissolving may in particular take place when the skin perspiration has a pH in the acidic range. On the other hand, skin perspiration in the alkaline range may leave the carrier film unaffected, for example when the carrier material is a polymer (for example polyester) with a thickness of less than 20 μm. The patch therefore controls the therapy as a function of the biochemical situation on the skin. The patch also therefore serves as a diagnostic aid: when the skin perspiration leads to parts of the patch being dissolved, this makes it possible to deduce that the skin perspiration has a pH in the acidic range and that there may therefore be inflammatory processes. In this case, for example, the patch may be turned over i.e. the coating of the opposite side is arranged closer to the skin.

The patch can be fixed on the skin in various ways, and the methods may also be combined with one another. At least one of the coatings may be provided with adhesive at places on the surface. The carrier material may be self-adhesive or have an adhesive applied to it, in which case the coating may be locally omitted and/or the carrier material may extend beyond the edge of the coating. It is, however, preferable for the patch to be combined with an additional second carrier layer. In this way, the patch itself can be made very thin (see below). The second carrier layer is, for example, a fabric layer provided surface-wide with adhesive, or another layer such as those conventionally used for tape dressings. In this case, the patch may optionally be adhesively bonded via one or other coating onto the second carrier layer, so that the layer which is not adhesively bonded remains free for contact with the skin. The second carrier layer protrudes, for example, at the edges of the dressing thus formed, so that it can be adhesively bonded onto the skin. Such a dressing or another dressing may be offered ready-made in retail trade or a pharmacy. The patch has, however, the advantage that it can also be delivered separately and therefore configured individually according to the application, so that the surface area of the patch and the method of fixing on the skin can vary.

The patch preferably has coatings on the opposite sides which respectively consist of an electrically conductive material, in particular metals (preferably gold, silver, copper and/or aluminum). In particular when the effect of an electrochemical battery is achieved by the patch (see above), new therapeutic effects can thereby be achieved: for example, the meridians known from Traditional Chinese Medicine can be connected not only electrically but via the patch acting as an electrochemical battery, when the patch is appropriately arranged on the skin. In order to promote the entry of skin perspiration into the carrier material, the coating to be arranged on the skin surface may have perforations so that the skin perspiration can penetrate directly into the carrier material. In the case of a thin coating (for example 10 nm to 100 nm thick), however, skin perspiration may also diffuse through the coating.

In a preferred embodiment of the patch, the mutually opposite sides of the carrier material configured as a carrier film are coated with a metal coating which is in particular continuous surface-wide. In this way, the thermal radiation emitted by the patient's skin is reflected back to a particularly high degree. This also applies when the metal coating on the side placed closer to the skin is very thin, i.e. it has the thickness mentioned elsewhere in this description. Even if thermal radiation still penetrates through the metal coating arranged closer to the skin surface and passes through the carrier material, this radiation component will be reflected back from the metal coating of the opposite side.

The second coating therefore has the advantage that a particularly large proportion of the thermal radiation is reflected back onto the skin, and the skin loses very little heat as a result. It has been shown that the healing processes in the living skin layers and underneath are thereby substantially promoted. This applies in particular when, as preferred, the patch is adhesively bonded to the skin circumferentially outside its circumferential edges, and no or only very little air exchange can therefore take place between the skin surface covered by the patch and the surroundings. Such an air seal furthermore has a regulating effect on a modified pH of the skin perspiration. In particular, the skin perspiration cannot evaporate or can evaporate only to a very small extent. In this way, on the one hand, no body heat is used up for the evaporation, and on the other hand a region of skin perspiration which has a healing effect is formed on the skin surface covered by the patch. Particularly in the case of silver or other metal coatings on the side of the patch lying closer to the skin, the skin perspiration is cleaned owing to the antibacterial effect and can re-enter the skin in a cleaned form, in particular without the addition of medicaments, and promote the healing process or the cosmetic process.

The patch may in particular be used in one of the following ways:
For the treatment of pressure points, i.e. when pressure has been exerted on the skin and the body regions lying underneath. Application is, in particular, also possible when blisters have formed. Particularly when the patch is made as thin as described elsewhere in this description, the patient can continue to wear clothing and shoes in the same way. This is because the thin patch is very unobtrusive and the pressure on the skin is not increased. Rather, the carrier layer configured as a carrier film and the coatings preferably protect the underlying skin surface and therefore act as an additional skin layer.
The patch is particularly suitable for use as an orthopedic patch. In particular, it is possible for the first time, even without adding medicinal compounds, to have a healing effect on orthopedic regions lying under the skin merely by applying a patch to the skin surface. In particular owing to the processes already described (thermal insulation, promoting the formation of perspiration, cleaning the skin perspiration), healing is promoted not only in the skin itself but also in the underlying orthopedic body regions (bone, cartilage, tendons and musculature). In particular, inflammations in joints, sprains and capsule injuries can be treated with the patch according to the invention.
According to Traditional Chinese Medicine (TCM), meridians running through the body can be electrically connected with the aid of the coatings configured continuously and therefore electrically conductively on the opposite sides. In particular, it is suitable to apply the patch on the foot or the hand where various meridians run close together. There are however other positions, for example on the arm, on the leg or on the back, where meridians running next to one another can be electrically connected together via the skin surface.
The patch can be used for diagnosis of whether an inflammation or other illness is present. To this end, the patch is applied onto the skin surface of the body position, particularly in the manner described above with an air seal from the surroundings. The skin perspiration caused in particular by the thermal insulation can interact with the coating which lies closer to the surface. Under particular circumstances mentioned elsewhere in this description, the skin perspiration can also interact with the second coating arranged on the opposite side. By this interaction or these interactions and any damage occurring to the carrier material, which is dependent on the chemical composition of the skin perspiration, it is possible to establish whether the skin perspiration is “normal” i.e. the covered body region is healthy, or whether changes from the normal state indicate a disorder or illness. The composition of the skin perspiration, which can be identified in particular from the pH of the composition, acts in particular on the metals preferably used as coating material. One essential process in this regard is the effect on the oxide layer formation which normally occurs on the surface of metals. Substances in the skin perspiration, in particular chlorine, prevent oxide layer formation. For example aluminum, which normally forms a stable protective layer of aluminum oxide, can therefore be attacked by other substances in the skin perspiration, i.e. the substances can react with the aluminum and therefore at least locally dissolve the aluminum coating. On the other hand, when using noble metals for the coating of the carrier film, depending on the composition of the skin perspiration, the skin perspiration can contribute to oxide layer formation which would otherwise not take place since noble metals do not react or react only to a small extent with atmospheric oxygen. The oxide layer which is formed can be identified in a straightforward way. The diagnostic capacity of the patch is particularly great when the patch is arranged on the skin surface with a seal against the ambient air. A “seal” is also intended to mean substantial impeding of air exchange which, for example, is achieved when the edge of the patch is adhesively bonded circumferentially to the skin as described above.

Other applications of the patch will be mentioned briefly below. In particular, scars with protruding adhesions of skin layers and hardenings can be treated by applying the patch. As a result, the skin becomes soft again in particular by promoting the blood supply, which also cosmetically has the effect that the scar becomes less conspicuous.

In particular by the thermally insulating and therefore blood-supply-promoting effect of the patch, but also by other processes which are stimulated by the patch, cell division is accelerated in the body region covered by the patch.

Particularly in the case of a continuous electrically conductive coating of metal over the surface of the carrier film, the entire region of the body covered by the patch is influenced in the same way. This is not achieved, for example, when using patches whose carrier material is a fabric or a nonwoven. With the patch according to the invention, owing to this equal treatment of the covered region in which there are preferably not only diseased subregions but also healthy subregions, harmonization takes place i.e. healthy subregions can influence diseased subregions and promote the healing. This harmonization takes place in particular when, with a uniformly thick carrier film, the mutually opposite sides are each provided with a metal coating and the infrared rays reflected back in the direction of the skin by the two metal coatings, as described above, and the infrared rays emitted by the skin can interfere in the same way irrespective of where on the patch these interference processes are observed. Therefore, according to the preferably constant thickness of the carrier film and according to the reflection properties of the metal coatings, a particular spectrum of infrared radiation is generated which acts as in the same way on the skin and the underlying regions everywhere in the region covered by the patch.

In particular by applying the patch onto the skin surface in the region of the breastbone, asthmatic phenomena can be treated, in particular those which have resulted from the effect of cold.

By applying the patch onto the skin surfaces which lie close to the airways of the body (for example nose, neck or breastbone region), diseases of the airways and therefore associated diseases can be treated.

By applying the patch onto wrinkles, the conspicuousness of these creases can be reduced. In particular, the skin in the region of the creases is supplied better with blood, is thereby smoothed and therefore appears less deep. Particularly for this application, but also in general, the patch can help to transport away toxins owing to the blood-supply-promoting effect and make the skin appear younger.

By applying the patch onto regions below which there are veins and/or lymphatic vessels, the flow through these vessels can be promoted.

It was mentioned above that the coating on one side of the carrier film may for example consist of gold and on the other side may consist of silver. Similar healing effects with the gold/silver combination can, however, also be achieved when silver-colored substances are used instead of silver and/or when gold-colored substances are used instead of gold. Silver-colored substances are in particular metals which gleam like silver, for example aluminum. Metals appearing similar to gold, i.e. gold-colored metals, are for example brass or anodized aluminum, which has been correspondingly colored (so-called gold eloxal).

According to a second aspect of the invention, the patch may also have only one coating, or two coatings as described above. In any case, the thickness of the film (carrier material and coating/s) will be less than 100 μm, preferably less than 20 μm and, for example, lies in the range of 12-15 μm.

A patch is therefore provided which entails the good treatment results of known patches but at the same time, owing to the extraordinarily thin configuration, does not cause any hindrance of wearing comfort or handling. In particular, wearing comfort or application are even made possible in tight footwear or compression stockings.

Refinements and configurations of the invention are specified in the dependent claims. An exemplary embodiment of the invention is represented in the drawing and will be explained in detail below.

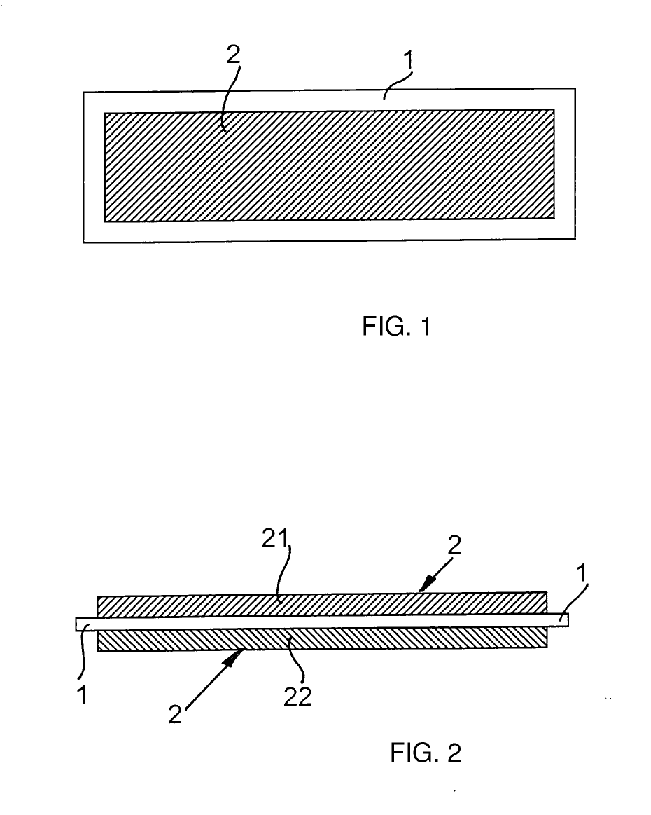

FIG. 1 shows the view of a patch according to the invention and

FIG. 2 shows the exaggerated side view of the patch represented in FIG. 1.

The patch selected as an exemplary embodiment is a metalized polyester film 1. It forms the carrier material for the patch. The film has for example a thickness of from 10 to 15 μm, and in the exemplary embodiment 12 μm. The film may be extensible. The extension properties in the length direction are for example from 100 to 130%; the transverse extension is for example about 100%.

The film 1 is provided with a coating 2. The coating 2 may be protective lacquering, preferably gold or silver protective lacquering. As a variant it may be a powder coating, which preferably likewise consists of silver or gold and is adhesively bonded to the film 1 or fastened to it in another way. The amount of coating applied is for example from 0.8 to 1.2 g/m². In the exemplary embodiment, the coating is applied on both sides of the film 1, the side denoted by “21” being provided with a gold coating and the other side denoted by “22” being provided with a silver coating. As an alternative to powder coating, the gold and/or silver may have been vacuum-evaporated (particularly in a high vacuum) onto the carrier material.

The film 1 may be configured to be self-adhesive. The adhesive is spread over the entire surface of the patch, so that extraordinarily good adhesion to the skin is achieved. As an alternative, however, the film may not have any externally lying adhesive but be fixed on the patient's skin by additional means (see above).

The film 1 is for example configured so that it is not air-permeable, or is air-permeable only to a small extent. In this way, after the film is fixed onto the skin, formation of perspiration is promoted in the region of the patch. The formation of perspiration contributes to an increased conductivity of the metalized film. Owing to the occurrence of perspiration in conjunction with air, gold or silver is dissolved from the film because the chloride present in perspiration prevents the formation of a protective layer on gold, so that the atmospheric oxygen in conjunction with the relatively low pH of the moist skin brings the gold or silver into solution. The conductivity of the patch on the skin is thereby increased, so that the healing process is additionally optimized. At the same time, sweating under the patch leads to softening of the skin which additionally improves the healing process, for example in the case of scars. Furthermore, bodily impurities are excreted by sweating, which leads to an improvement of the metabolism. Said effects also mean that skin perspiration can penetrate into the carrier material so that the coating on the side lying further away from the skin also delivers a therapeutic effect.

The patch according to the invention is preferably made very thin (for example at most 20 micrometers thick). It is therefore possible to wear the patch even in regions in which the known patches would lead to a compromise of wearing comfort. This applies especially in shoes, particularly in the region of the toes, or in the case of ski boots in the region of the known pressure points on the ankle, the shinbone etc.

The same also applies when wearing compression stockings, for example for venous or lymphatic weakness. In said regions, there is no space between the skin and the footwear or stocking, so that the known patches always “obtrude” and the pressure on the point in question is increased even further. This is avoided with the patch according to the invention; owing to its extraordinarily thin configuration, the patch is scarcely perceptible.

Owing to the thin configuration of the patch according to the invention, the handling of compression stockings is also substantially simplified since with these, when using conventional patches, the latter normally become detached or rubbed off when the stocking is pulled over. For this reason, the patches lose their intended positioning so that the effect cannot take place properly. With the wafer-thin patches according to the invention, this problem is avoided since the compression stockings can be pulled over the patch unimpeded; with the self-adhesive configuration over the entire surface of the patch, good bonding properties are furthermore produced which allow adhesion to the skin when the stockings are pulled over. The good bonding properties also avoid detachment over a prolonged wearing period, so that the patch according to the invention can readily be used even over several days.

Providing the coating on both sides of the patch, in particular when using different coatings on the two sides of the patch, for example the coating of gold on one side and silver on the other side according to the exemplary embodiment, provides the opportunity to select a treatment type according to the application. For instance, the side coated with silver will generally be arranged closer to the skin when the intention is to achieve a cooling and anti-inflammatory effect, which leads to soothing and therefore contributes to the reduction of swelling. The gold-coated side will be arranged closer to the skin when heating is required on the side in question since, as is known, gold stimulates the blood supply which leads to increased blood circulation that triggers a heating effect. Two different treatment and application cases are therefore combined in the patch according to the invention, so that just one patch needs to be stocked in order to be able to treat both cases. The choice is made directly before the use of the patch. For the user, this offers the advantage that two treatment cases can be catered for with only one patch to be taken with them.

As described above, however, the coating lying further away from the skin also contributes to the therapy, in particular when the carrier material is made very thin or dissolves in the course of use.

1-15. (canceled)

16. A patch to be adhered to the skin for medical, cosmetic, and/or orthopedic purposes, the patch comprising:
a film consisting of a carrier material and having two sides; and
a therapeutically effective coating disposed on each of said two sides of said film, said coating being selected from the group consisting of a gold coating, a gold-colored metal coating, a silver coating, and a silver-colored metal coating;
said film and said coatings together having a thickness of less than 100 μm; and
an adhesive disposed circumferentially at a peripheral edge and/or on a carrier layer outside the circumferential edge of said film, for sealing a skin region covered by said film against ambient air.

17. The patch according to claim 16, wherein said film and said coatings together have a thickness of less than 20 μm.

18. The patch according to claim 16, wherein said film is a stretchy, extensible film.

19. The patch according to claim 16, wherein said coating on one side of said film is a silver coating or a silver-colored metal coating, and said coating on the opposite side of said film is a gold coating or a gold-colored metal coating.

20. The patch according to claim 16, wherein said coating on one side of said film is a protective lacquering.

21. The patch according to claim 16, wherein said coating on one side of said film is a powder coating.

22. The patch according to claim 16, wherein said coatings on the opposite sides of said film consist of different materials.

23. The patch according to claim 16, wherein said coatings on the opposite sides of said film respectively consist of or contain an electrically conductive material.

24. The patch according to claim 23, wherein at least one of said electrically conductive materials is a noble metal.

25. The patch according to claim 16, wherein said carrier material consists of a polymer.

26. The patch according to claim 25, wherein said carrier material is a polyester.

27. The patch according to claim 16, wherein said coating on at least one side of said film has a thickness of less than 5 μm.

28. The patch according to claim 27, wherein said coating on said at least one side of said film has a thickness of less than 500 nm.

29. The patch according to claim 16, wherein said adhesive is configured to also adhesively bond the patch onto the skin of a patient.

33. A method for producing a dermal patch for medical, cosmetic, and/or orthopedic purposes, the method which comprises:
providing a film of a carrier material with two sides, and providing the film with a therapeutically effective coating on both sides thereof.

34. A method for producing a patch according to claim 16, the method which comprises:
providing a film of a carrier material with two sides, and coating the film with a therapeutically effective coating on both sides thereof, by forming a protective lacquering on the film, by producing a powder coating on the film, or by vapor-depositing silver and gold, respectively, on the sides of the film to produce the patch according to claim 16.

35. A method for producing a patch to be adhered to the skin for medical, cosmetic, and/or orthopedic purposes, the method which comprises:
providing a film of a carrier material with one side and an opposite side;
providing the one side of the film with a coating of gold or a gold-colored metal;
providing the opposite side of the film with a coating of silver or a silver-colored metal;
to produce an assembly of the film and two coatings having a total thickness of less than 100 μm;
providing an adhesive circumferentially at a peripheral edge and/or on a carrier layer outside the circumferential edge of the film, for sealing a skin region covered by the film against ambient air when the patch is adhered to the skin of a patient.
